(19) United States
(12) Patent Application Publication
(10) Pub. No.: US 2008/0310581 A1
(43) Pub. Date: Dec. 18, 2008

(54) Medical diagnostic system

(76) Inventors: Ute Feuerlein, Erlangen (DE); Harald Nutzel, Forchheim (DE)

Correspondence Address: HARNESS, DICKEY & PIERCE, P.L.C., P.O.BOX 8910, RESTON, VA, 20195, US

(21) Appl. No.: 12/155,049

(22) Filed: May 29, 2008

(30) Foreign Application Priority Data
May 31, 2007 (DE) 10 2007 025 399.2

(51) Int. Cl. A61B 6/00

(52) U.S. Cl. 378/4; 600/431

(57) ABSTRACT

In a method for operating a medical diagnostic system including an irradiating examination machine and a contrast agent supplying machine, at least one operational parameter is assigned to the examination machine and at least one application parameter is assigned to the contrast agent supplying machine. In at least one embodiment disclosed herein, the operational parameters and the application parameters are concurrently, or even simultaneously, displayed via a display apparatus.

### PRIORITY STATEMENT

The present application hereby claims priority under 35 U.S.C. §119 on German patent application number DE 10 2007 025 399.2 filed May 31, 2007, the entire contents of which is hereby incorporated herein by reference.

### FIELD

Embodiments of the invention generally relate to a method for operating a medical diagnostic system and/or an apparatus suitable for carrying out the method. In at least one embodiment, the medical diagnostic system operates by irradiation, with a contrast agent being used for the irradiating examination, which in particular uses ionizing rays.

### BACKGROUND

DE 102 30 877 A1 discloses a magnetic resonance imaging machine having a device for graphically planning contrast agent supported angiographic measurements. This device takes account of the temporal profile of venous contrast agent enrichment in a test bolus measurement in order to permit a graphic display in form of a test bolus chart. In addition to the test bolus chart, an angiomap is provided, which shows individual measurements in the form of measurement bars in relation to a time axis and can be used, inter alia, to determine the number of measurements.

DE 10 2004 003 371 A1 discloses a method for operating two machines, namely a computed tomography scanner and an injector. The two machines communicate with each other via a data interface in order to adjust their operation to each other. Such an adjustment means, for example, that when one of the machines is started, a check whether the other machine is operational is automatically carried out. It is likewise provided to transmit a malfunction of one machine to the other machine and to display it there.

DE 10 2005 024 323 A1 discloses a method for determining operational parameters for an x-ray machine. In this method, projections, which are the basis for a series of contrast agent measurements, are acquired at least at two different scanning positions. The measurements are carried out at the respective scanning position until a point in time at which the contrast agent is detected at the related scanning position. Subsequently the operational parameters of the x-ray machine are set, taking the determined times into account.

By way of example, x-ray examinations using a contrast agent can be carried out in the form of an angiocontrast examination, arterial-contrast examination or bronchial-contrast examination. The use of contrast agents is particularly important in the case of examinations of the liver. Computed tomography examinations of the liver can generally be subdivided into four phases: namely the native phase (without contrast agent), the early phase (arterial phase), the portal venous phase and the equilibrium.

In the early phase, the administered contrast agent becomes enriched in the liver via the hepatic artery. During the second contrast agent phase, the portal venous phase, there is an invasion of contrast agent via the hepatic portal vein. Finally, in the equilibrium, the contrast agent is evenly distributed in the whole body.

Details on this are disclosed, for example, in the dissertation “Optimierung der bolusgetriggerten Spiral Computertomographie der Leber und Vergleich zum empirischen Bolustiming” [Optimizating bolus-triggered spiral computed tomography of the liver and comparison with empirical bolus timing] (by Carsten Vogelsang, Julius-Maximilians-Universität [Julius Maximilian University] Würzburg, Institut for Röntgendiagnostik [Institute for x-ray diagnostics], December 2005). The dissertation discusses so-called bolus tracking in great detail.

In this method, a density rise is detected in the tissue to be examined after the contrast agent has been dispensed, with the variation in density being tracked with the so-called low-dose method with decreased exposure to radiation. When there is a sufficient density rise in the target organ, the actual data recording, performed by the spiral computed tomography scanner, is triggered by a machine induced start delay.

### SUMMARY

In at least one embodiment of the invention, possibilities are specified, improved by comparison with the prior art, for operating a medical diagnostic system including an imaging irradiating machine and a machine for supplying a contrast agent.

An irradiating medical machine and a machine for supplying a contrast agent to the object to be examined are provided in at least one embodiment, for the imaging examination of an object, in particular a patient. The examination machine is operated by at least one operational parameter which is set automatically or by the user, while the contrast agent supplying machine is assigned at least one application parameter. Without loss of generality, all the parameters relating to the examination machine are referred to in the following as operating parameters, and the parameters relating to the contrast agent supplying machine are referred to as application parameters, for the purpose of being able to distinguish between them easily. At least one operating parameter and at least one application parameter are simultaneously displayed by means of a display apparatus. The display apparatus is connected to a data processing machine which is linked both to the examination machine and to the contrast agent supplying machine. The imaging examination machine is preferably a computed tomography machine suitable for spiral computed tomography. By way of example, it can likewise be a magnetic resonance machine or an angiography machine.

The display apparatus preferably also displays the planned and/or actual temporal variation, for example, in one or more operating parameters and one or more application parameters. Keywords to be mentioned in this context are bolus shaping, ratio and pressure limit. A graphic display, especially in diagram form, is particularly advantageous. By way of example, the possibility is provided of showing the start times and the durations of individual data acquisition phases to be undertaken by the examination machine, also referred to as scans in the case of a computed tomography scanner and, simultaneously, of showing—inserted in one and the same graphic—the flow and the volume of a contrast agent dispensed by the contrast agent supplying machine. In a particularly user-friendly refinement, it is possible to change parameter settings which are visible on the display apparatus and which relate to at least one of the machines of the diagnostic system by means of so-called graphic manipulation. By changing a value displayed on a screen, for example by clicking on it and displacing it with a computer mouse, the corresponding actual parameter setting is also changed, preferably in real-time.

The acquisition of data with a medical imaging machine during temporally separated phases, described by the term “multiphase protocol” for short, is of great importance particularly in connection with examinations of the liver. In this context, reference is made to the dissertation by C. Vogelsang mentioned initially.

At least one embodiment of the inventive method is particularly suitable for visualizing bolus tracking, with both parameters of the imaging machine and parameters of the contrast agent application being shown continuously on one and the same screen display in a clear fashion. This is also called timeline visualization because the temporal change is taken into account, either in the course of planning or—during an examination—in real-time.

If required, a plurality of y-axes is integrated into a single diagram, the x-axis of which represents the time axis, the y-axes firstly being assigned to different parameters of the examination machine (operational parameter) and secondly being assigned to the contrast agent application (application parameter). It is not necessarily the case here that only continuously variable values are displayed on the y-axis. Rather, in at least one embodiment, it can also be advantageous in individual cases to insert discrete values into a two dimensional diagram (x-y diagram), which provide, for example, on/off information. By way of example, such on/off information relates to being informed whether a scan with the imaging diagnostic machine, generally referred to as examination machine, is being carried out during a particular period.

The diagnostic system designed according to at least one embodiment of the invention also provides a particularly user-friendly planning tool for the preparation of an imaging examination supported by a contrast agent. Parameters of the examination, for example the volume flow and the duration of the supply of a contrast agent or saline, can directly be set or changed using a graphical user interface. The same is true for the parameters of the imaging examination machine which can be set, in particular for the start times and durations of the individual phases of a scan, which are also referred to as examination stages. Generally this data recorded in the individual examination stages is influenced by the applied contrast agent. A test bolus can also be included in the method.

According to one advantageous development, parameter settings or changes undertaken by the user are automatically evaluated; in particular they are subject to an automatic plausibility check. By way of example, it can be provided to output a warning message if a user release is very likely to be erroneous according to an automatic control of the input data. A recommended standard setting of a parameter can be shown not only in this case. Moreover, it is possible to automatically change the parameter settings or suggested parameter settings if a different parameter is adjusted by the user. Thus, the possibility of semi-automatic setting of parameters is provided. If actually chosen parameters differ from preset parameters then such deviations are advantageously displayed graphically. In an analogous manner, a visualization of the relations between different parameter changes is also advantageous. By way of example, when an operational parameter is set to a new value, an application parameter changes as well. In this case, not only the respectively current parameter settings are shown, but also the relations between the parameters and the parameter changes of the different machines, namely the imaging examination machine, on the one hand, and the contrast agent supplying machine, on the other.

Overall, the diagnostic system provides not only a clear visualization with a largely intuitive usability, but also a clearly structured documentation of contrast agent examinations. Furthermore, the common administration and visualization of scan parameters and injection parameters offers particularly good preconditions for repeating contrast agent examinations with reproducibly set parameters as many times as desired. The risk of erroneous setting of parameters is also minimized thereby. The optimized ability to evaluate the image data obtained by the examination machine in turn substantially contributes to a decrease in the exposure of the patient to the contrast agent and/or radiation.

In particular, an advantage of at least one embodiment of the invention is that the planning, conducting and evaluating of an irradiating medical examination supported by a contrast agent is substantially eased by the use of a data processing system which is linked to both the irradiating machine and the contrast agent supplying machine, with an increased operating comfort being achieved by one and the same display apparatus showing both parameters of the irradiation and parameters of the contrast agent supply, and relations between the different parameters, if applicable.

### BRIEF DESCRIPTION OF THE DRAWINGS

In the following text, an example embodiment of the invention is explained in more detail with the aid of drawings, in which:

FIG. 1 shows a symbolic display of a medical diagnostic system, and

FIG. 2 shows a diagram of parameter settings of a method which can be carried out with the diagnostic system in accordance with FIG. 1.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

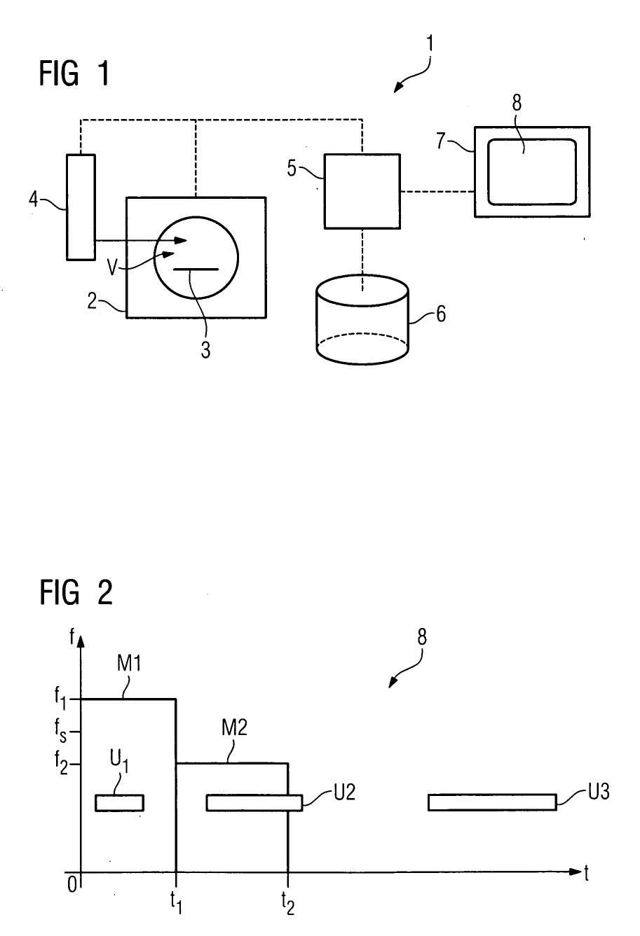

A medical diagnostic system 1 exemplified in a schematic block diagram in FIG. 1 has an imaging examination machine 2 which is suitable for an irradiating examination of a patient, for example a computed tomography machine. Generally the examination machine 2, also referred to as a scanner, can be any medical apparatus whose function can be supported by the use of a contrast agent to be supplied to the patient (not illustrated), be it a positive contrast agent, a negative contrast agent or a combination of a positive and a negative contrast agent.

The tissue of the patient lying on the patient couch 3, who is to be examined, in particular, by way of spiral computed tomography is located in an examination volume V during the examination.

A contrast agent is administered to the patient prior to the actual imaging examination, for example a non-ionic contrast agent including iodine or barium, by way of an injector 4, which is also referred to as a contrast agent supplying machine. The contrast agent supplying machine 4 and the examination machine 2 are connected to a data processing machine 5 using data systems technology, as indicated by dashed lines. In this case, the data-technology connections can be implemented as a hard wired and/or wireless connection. By way of example, the CANOpen standard CIA425 (Class 4) is suitable for data exchange.

The data processing machine 5 is liked to a data memory 6 which can be part of the data processing machine 5 or a component of a larger data processing network (not illustrated in any more detail). Data from contrast agent examinations carried out by the diagnostic system 1 can be archived in the data memory 6. Due to a clearly structured documentation of examinations carried out previously, settings undertaken in these can easily be reproduced at any time. Furthermore, the data processing machine 5 is connected to a display apparatus 7, in particular a commercially available screen.

A screen display 8 which can be shown on the screen 7 is illustrated in FIG. 2 by way of example. In the manner of a diagram, parameters of the examination machine 2 and of the contrast agent supplying machine 4 are simultaneously shown here as a function of time t (the time interval displayed on the x-axis is, for example, 150 seconds).

The application of a contrast agent starts at time t=0, with the flow rate (volumetric flow) f being specified in ml/s. Up until the time t₁, a preferably constant volumetric flow f₁ of a few ml per second (e.g. 4 ml/s) is supplied. The total amount of the applied contrast agent, referred to as volume M1, is clearly visualized as a rectangular area in the diagram.

Saline is supplied from the time t₁ until the time t₂ at a flow rate f₂, which is less than the flow rate f₁ in the exemplary embodiment, and also slightly less than a standard flow rate referred to by fₛ. The total amount of applied saline is referred to by M2.

In the example embodiment according to FIGS. 1 and 2, an examination of the liver is being carried out by way of the diagnostic system 1. In this case, a multiphase protocol is selected for the operation of the computed tomography scanner 2, the protocol being visualized, just like the application of different amounts of contrast agent and saline on the screen display 8. In contrast to the visualization of the application parameters, that is to say the parameters of the contrast agent supplying machine 4 including a quantitative display of selected parameters (in this case: flow rates f₁, f₂; volumes M1,M2), the visualization of the operational parameters of the examination machine 2 only presents digital information as to at what times an imaging measurement is carried out.

The entire data acquisition carried out with the imaging examination machine 2 is divided into three temporally separated stages, the examination stages U1,U2,U3. The first examination stage U1, which lies completely within the time period of the application of the contrast agent, is the so-called monitoring phase. In this stage U1, a rise in the contrast agent in the tissue to be examined is automatically detected. In this case, the measurement region can lie either directly in the liver or in the aorta. In the former case this is referred to as direct triggering, in the latter case it is referred to as indirect triggering.

The rise in contrast agent concentration initiates the second examination stage U2, the arterial phase, independently of the precise configuration of the triggering. In the illustrated example, this phase U2, in which the administered contrast agent is enriched in the liver via the hepatic artery, starts during the supply of saline and finishes a few seconds after the supply of saline has been terminated. The duration of the arterial phase U2 of the examination of the liver is substantially longer than the duration of the monitoring phase U1, as likewise clearly emerges from FIG. 2.

A pause having a longer duration than the phase U2 follows the arterial phase U2. The third examination stage U3, namely the portal venous phase, in, which there is an invasion of the applied contrast agent via the hepatic portal vein, starts following this pause, approximately 120 s after the time t=0.

By way of example, the sequence according to FIG. 2 can be a planned scenario. Using this as a starting point, the operator of the medical diagnostic system 1 has the option of inserting, displacing or deleting examination stages (scans) and/or injection phases (relating to the contrast agent and/or the saline), depending on the requirements of the individual case. In a corresponding manner it is possible to change the flow rate of the contrast agent or saline. By way of example, for this purpose, the operator places a marker in the diagram on the value to be changed (f₁ or f₂), and displaces the marker on the screen display 8 in the desired direction. In any case, the flow of the contrast agent which typically varies with time is immediately depicted in terms of a so-called bolus shaping. An automatic plausibility check is simultaneously carried out by the data processing machine 5. The operator is thus put into the position of optimizing the parameter settings of the diagnostic system 1 in a particularly comfortable manner and with a very wide-spread elimination of sources of error.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for operating a medical diagnostic system including an irradiating examination machine and a contrast agent supplying machine, the method comprising:
assigning at least one operational parameter to the irradiating examination machine;
assigning at least one application parameter to the contrast agent supplying machine;
concurrently displaying the at least one operational parameter and the at least one application parameter, via a display apparatus of a data processing machine operationally linked both to the irradiating examination machine and to the contrast agent supplying machine, parameter settings being visible on the display apparatus and relating to at least one of the machines being automatically changed during graphical manipulation; and
visualizing via the display apparatus, in the case of a change in at least one parameter of at least one of the irradiating examination machine and the contrast agent supplying machine, both a new setting of the at least one parameter of at least one of the irradiating examination machine and the contrast agent supplying machine and a parameter change of the other one of the irradiating examination machine and the contrast agent supplying machine.

2. The method as claimed in claim 1, wherein a temporal variation in the at least one operational parameter of the irradiating examination machine and in the at least one application parameter of the contrast agent supplying machine are displayed.

3. The method as claimed in claim 2, wherein the display of the temporal variation in the different parameters is carried out in diagram form.

4. The method as claimed in claim 1, wherein start times of a plurality of examination stages, to be carried out by the irradiating examination machine, are separated from one another in time and are influencable by the supply of contrast agent, are shown via the display apparatus.

5. The method as claimed in claim 4, wherein a duration of individual examination stages is shown via the display apparatus.

6. The method as claimed in claim 4, wherein a temporal relationship between the setting of application parameters of the contrast agent supplying machine and the progress of the examination stages is shown via the display apparatus.

7. The method as claimed in claim 4, wherein the examination stages include a monitoring phase which prepares the imaging examination.

8. The method as claimed in claim 1, wherein a flow and a volume of a contrast agent dispensed by the contrast agent supplying machine are displayed concurrently in graphical form via the display apparatus.

9. The method as claimed in claim 1, wherein a comparison between a preset standard parameter and an application parameter actually selected for the examination is displayed via the display apparatus.

10. A medical diagnostic system to carry out the method as claimed in claim 1.

11. The method of claim 1, wherein the concurrent display includes simultaneous display.

12. The method as claimed in claim 5, wherein a temporal relationship between the setting of application parameters of the contrast agent supplying machine and the progress of the examination stages is shown via the display apparatus.

13. The method as claimed in claim 5, wherein the examination stages include a monitoring phase which prepares the imaging examination.

14. The method as claimed in claim 6, wherein the examination stages include a monitoring phase which prepares the imaging examination.

15. The method as claimed in claim 12, wherein the examination stages include a monitoring phase which prepares the imaging examination.

16. The method of claim 8, wherein the concurrent display includes simultaneous display.

17. A medical diagnostic system, comprising:
a device to assign at least one operational parameter to an irradiating examination machine;
a device to assign at least one application parameter to a contrast agent supplying machine; and
a display apparatus, operationally linked both to the irradiating examination machine and to the contrast agent supplying machine, to concurrently display the at least one operational parameter and the at least one application parameter, parameter settings being visible on the display apparatus and relating to at least one of the machines being automatically changed during graphical manipulation, and to visualize, in the case of a change in at least one parameter of at least one of the irradiating examination machine and the contrast agent supplying machine, both a new setting of the at least one parameter of at least one of the irradiating examination machine and the contrast agent supplying machine and a parameter change of the other one of the irradiating examination machine and the contrast agent supplying machine.

18. The medical diagnostic system of claim 17, wherein the concurrent display includes simultaneous display.
